(19) United States
(12) Patent Application Publication
(10) Pub. No.: US 2025/0201437 A1
(43) Pub. Date: Jun. 19, 2025

(54) GENERATING METHOD, SYSTEM, AND DEVICE OF SCANNING IMAGE, AND COMPUTER-READABLE STORAGE MEDIUM

(71) Applicant: Shanghai United Imaging Healthcare Co., Ltd., Shanghai (CN)

(72) Inventors: Cheng Ni, Shanghai (CN); Can Liao, Shanghai (CN); Wei Zhang, Shanghai (CN); Jingjie Zhou, Shanghai (CN); Hongcheng Yang, Shanghai (CN); Xiaohua Zhu, Shanghai (CN); Li Wang, Shanghai (CN)

(21) Appl. No.: 18/981,774

(22) Filed: Dec. 16, 2024

(30) Foreign Application Priority Data
Dec. 15, 2023 (CN) 202311736649.5

(51) Int. Cl. G21K 1/04

(52) U.S. Cl. CPC G21K 1/043

(57) ABSTRACT

A generating method, a system, and a device of a scanning image, and a computer-readable storage medium are provided. The method includes: scanning different positions of a target object, respectively, by changing a passing region of an imaging beam emitted from a scanning imaging source, obtaining a plurality of scanning data of the target object, and obtaining a scanning image corresponding to the target object based on the plurality of scanning data of the target object.

### CROSS-REFERENCE TO RELATED APPLICATION

The present disclosure claims priority to Chinese patent applications No. 202311736649.5, filed on Dec. 15, 2023, titled “GENERATING METHOD, SYSTEM, AND DEVICE OF SCANNING IMAGE, AND COMPUTER-READABLE STORAGE MEDIUM”, the content of which is hereby incorporated by reference in its entirety.

### TECHNICAL FIELD

The present disclosure generally relates to the field of medical image, and in particular, to a generating method, a system, and a device of a scanning image, and a computer-readable storage medium.

### BACKGROUND

With development of computer technologies, X-ray tomography is provided, which is divided into fan beam tomography (Fan-beam CT) and cone beam tomography (Cone-beam CT) imaging technologies. A width of a detector of the Fan-beam CT generally varies from 1 cm to 16 cm along an axial direction. Therefore, to meet a requirement of a relatively long scanning length (greater than 20 cm) in some scenarios, the Fan-beam CT usually completes scanning by a continuous (helical scanning) or stepwise (tomographic scanning) motion of a bed. The Cone-beam CT may use a large-size flat panel detector, and in a case that a bed is still, a tomographic image of a large scanning range may be obtained by scanning via rotating a ray source and a detector for a circle.

However, due to a problem of X-ray scattering in a large field, a problem of a collecting rate of the flat panel detector, and a problem of a signal-to-noise ratio of the flat panel detector, image quality of the Cone-beam CT is often inferior to that of the Fan-beam CT. The scanning length of the Fan-beam CT does not meet the requirement, and motion artifacts are also more likely to be generated for imaging a moving object. Therefore, the X-ray tomography in the related art has poor imaging effects of scanning a target object. In addition, the Cone-beam CT needs to move the flat panel detector and an imaging source to complete imaging. Therefore, there is a relatively high requirement for a motion control system and algorithm, an anti-collision system and algorithm of the flat panel detector and the imaging source, and the cost is relatively high. Furthermore, a space for motion of the detector and a ray source needs to be reserved.

### SUMMARY

According to various embodiments of the present disclosure, a generating method, a system, and a device of a scanning image, a computer-readable storage medium, and a computer program product are provided.

In a first aspect, a generating method of a scanning image is provided, including: scanning different positions of a target object, respectively, by changing a passing region of an imaging beam emitted from a scanning imaging source, obtaining a plurality of scanning data of the target object, and obtaining a scanning image corresponding to the target object based on the plurality of scanning data of the target object.

In a second aspect, a generating system of a scanning image is further provided in the present disclosure, including a scanning device and a computer device. The scanning device is configured for scanning different positions of a target object, respectively, by changing a passing region of an imaging beam emitted from a scanning imaging source, and obtaining a plurality of scanning data of the target object. The computer device is configured for obtaining a scanning image corresponding to the target object based on the plurality of scanning data of the target object.

In a third aspect, a generating apparatus of a scanning image is further provided in the present disclosure, including an image scanning module and an image combining module. The image scanning module is configured for scanning different positions of a target object, respectively, by changing a passing region of an imaging beam emitted from a scanning imaging source, and obtaining a plurality of scanning data of the target object. The image combining module is configured for obtaining a scanning image corresponding to the target object based on the plurality of scanning data of the target object.

In a fourth aspect, a generating device of a scanning image is further provided in the present disclosure, including a scanning imaging source, an imaging beam collimating apparatus, a detector, and a processor. The imaging beam collimating apparatus is configured for changing a passing region of an imaging beam emitted from the scanning imaging source, so that the imaging beam scans different positions of a target object, respectively. The detector is configured for receiving the imaging beam, and obtaining a plurality of scanning data corresponding to the different positions of the target object based on the imaging beam. The processor is configured for obtaining a scanning image corresponding to the target object based on the plurality of scanning data of the target object.

In a fifth aspect, a radiation delivery system is further provided in the present disclosure, including the foregoing generating device of the scanning image and a radiation delivery device. The radiation delivery device is configured for performing radiation delivery to the target object based on the scanning image of the target object.

In a sixth aspect, a computer device is further provided in the present disclosure, including a memory and a processor. The memory stores a computer program, and the computer program is executed by the processor to implement following steps: scanning different positions of a target object, respectively, by changing a passing region of an imaging beam emitted from a scanning imaging source, obtaining a plurality of scanning data of the target object, and obtaining a scanning image corresponding to the target object based on the plurality of scanning data of the target object.

In a seventh aspect, a computer-readable storage medium is further provided in the present disclosure, a computer program is stored on the computer-readable storage medium, and the computer program is executed by a processor to implement following steps: scanning different positions of a target object, respectively, by changing a passing region of an imaging beam emitted from a scanning imaging source, obtaining a plurality of scanning data of the target object, and obtaining a scanning image corresponding to the target object based on the plurality of scanning data of the target object.

In an eighth aspect, a computer program product is further provided in the present disclosure, including a computer program. The computer program is executed by a processor to implement following steps: scanning different positions of a target object, respectively, by changing a passing region of an imaging beam emitted from a scanning imaging source, obtaining a plurality of scanning data of the target object, and obtaining a scanning image corresponding to the target object based on the plurality of scanning data of the target object.

Details of one or more embodiments of the present disclosure are set forth in the following accompanying drawings and description. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a schematic diagram of an application environment of a generating method of a scanning image in an embodiment.

FIG. 2 is a flowchart of a generating method of a scanning image in an embodiment.

FIG. 3 is a flowchart of a method of obtaining scanning data of an object in an embodiment.

FIG. 4 is a flowchart of a first method of scanning a target object in an

embodiment.

FIG. 5 is a flowchart of a second method of scanning a target object in an embodiment.

FIG. 6 is a flowchart of a third method of scanning a target object in an embodiment.

FIG. 7 is a flowchart of a fourth method of scanning a target object in an embodiment.

FIG. 8 is a flowchart of a fifth method of scanning a target object in an embodiment.

FIG. 9 is a flowchart of a continuous scanning procedure in an embodiment.

FIG. 10 is a schematic diagram of a method of changing an imaging beam passing region by a movable collimator in an embodiment.

FIG. 11 is a schematic diagram of a method of changing an imaging beam passing region by a movable collimating array in an embodiment.

FIG. 12 is a schematic diagram of a method of changing an imaging beam passing region by an imaging source with a fly focus in an embodiment.

FIG. 13 is a schematic diagram of a method of changing an imaging beam passing region by an imaging source array in an embodiment.

FIG. 14 is a schematic diagram of a method of changing an imaging beam passing region by two imaging sources in an embodiment.

FIG. 15 is a block diagram of a generating apparatus of a scanning image in an embodiment.

FIG. 16 is an internal schematic diagram of a computer device in an embodiment.

### DETAILED DESCRIPTION OF THE EMBODIMENT

To make objectives, technical solutions, and advantages of the present disclosure clearer, the following further describes the present disclosure in detail with reference to the accompanying drawings and embodiments. It should be understood that the specific embodiments described herein are merely used to explain the present disclosure, and are not intended to limit the present disclosure.

A generating method of a scanning image provided in an embodiment of the present disclosure may be applied to an application environment shown in FIG. 1. A scanning device 102 may be in communication with the server 104 by a network. A data storage system may store data that needs to be processed by the server 104. The data storage system may be integrated on the server 104, or may be placed on a cloud or another network server. The server 104 may be implemented by an independent server or a server cluster including a plurality of servers.

In addition, although CT imaging is mainly used as an example of the generating method of a scanning image (which may also be referred to a scanning imaging method) in the specification of the present disclosure, it may be learned that the content of the present disclosure may be used in other types of generating methods of scanning images, such as DR (Digital Radiograph) imaging, nuclear magnetic resonance imaging, and the like.

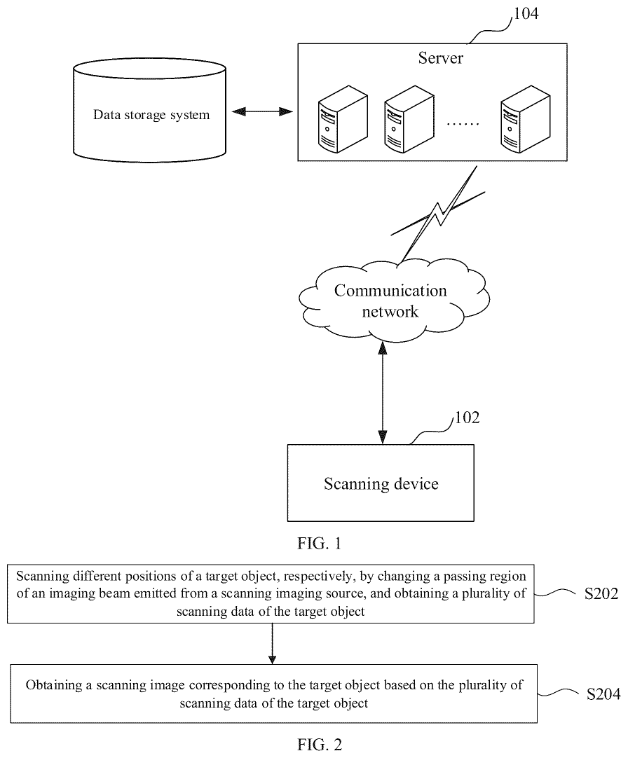

In an exemplary embodiment, referring to FIG. 2, a generating method of a scanning image is provided for description by an example in which the method is applied to the server in FIG. 1, and the method includes following step 202 to step 204.

Step 202 includes that scanning different positions of a target object, respectively, by changing a passing region of an imaging beam emitted from a scanning imaging source, and obtaining a plurality of scanning data of the target object.

The scanning imaging source may be a portion of a scanning device that emits the imaging beam, for example, a portion of a scanning device such as a CT machine or a DR machine that emits an X-ray.

The passing region may be a region that the imaging beam emitted by the scanning imaging source can pass through or penetrate, for example, a hole or a gap of an imaging beam collimating apparatus. Herein, the change of the passing region may include any form of change, and as a non-limiting example, may include a change of any one or more of a location, a shape, or an orientation of the passing region. The imaging beam collimating apparatus may be an imaging beam direction selector, configured to select a direction of the imaging beam that needs to be projected onto a detector, for example, by means of a movement of the imaging beam collimating apparatus, opening or closing of the hole or the gap of the imaging beam collimating apparatus, or a deformation of the hole or the gap of the imaging beam collimating apparatus, an imaging beam with a specific direction, a specific shape, or a specific position may be allowed selectively to emit from the imaging beam collimating apparatus through the passing region, and to direct to the target object.

The target object may be a person or an object that receives scanning of the scanning device.

The scanning data of the target object may include a scanning signal obtained based on an imaging beam that arrives on the detector after passing through the imaging beam collimating apparatus, and/or a scanning image reconstructed based on the scanning signal, or the like. In addition, the scanning data of the target object may be a sub-image, or may be a final image obtained after reading a signal of a plate directly and processing the signal.

Specifically, when it is ensured that the scanning imaging source has sufficient flexibility, imaging beams may be emitted along different directions and angles. By adjusting a position and an angle of the imaging source, different positions of the target object may be scanned. By accurately controlling the passing region of the imaging beam, it is ensured that a specific region of the target object is irradiated, and scanning information of a corresponding position is obtained. In this way, scanning may be repeatedly performed for multiple times, and the position and the passing region of the imaging source may be adjusted each time, so that scanning sub-images of the target object at different positions may be finally obtained, thereby implementing comprehensive and detailed scanning of the target object.

In an unlimited embodiment, when a CT scanning device with X-ray is considered, the imaging beam collimating apparatus may be in operation with the scanning imaging source. When the target object is detected, the device may first place the imaging beam collimating apparatus and the scanning imaging source in an initial position relationship, and perform overall scanning on the target object to acquire a whole-body CT image. Then, the position relationship may be adjusted automatically, i.e., the device may move the imaging beam collimating apparatus and the scanning imaging source to a new position, and perform scanning again. This process may be repeated until all angles of the target object are covered. Data generated by each scanning may be recorded to form a whole-body 3D CT image of the target object.

Step 204 includes that obtaining a scanning image corresponding to the target object based on the plurality of scanning data of the target object.

The scanning image of the target object may be obtained by scanning of a scanning device on the target object.

As an unlimited example, the plurality of scanning data of the target object may be combined to obtain the scanning image of the target object. Specifically, in a case that the scanning data of the target object is a scanning image (which may also be referred to a sub-image), a process of combining the plurality of scanning data of the target object may include image registration and superposition. Sub-images may be aligned to a unified coordinate system by an image registration algorithm, to ensure that the sub-images are in the same spatial position. This may involve feature point matching or other registration techniques. Furthermore, the aligned sub-images may be superposed, and a complete scanning image of the target object may be obtained by a pixel-level weighted average or other image combination method.

As another unlimited example, the plurality of scanning data of the target object may be reconstructed to obtain a scanning image of the target object. Specifically, in a case that the scanning data of the target object is a scanning signal, the scanning signal may be processed (for example, filtered, de-duplicated, de-noised, or the like) to obtain a processed signal required for reconstruction, and then image reconstruction may be performed based on the processed signal to obtain a complete scanning image of the target object.

As an example, the obtained scanning image of the target may be a two-dimensional image, a three-dimensional image, a four-dimensional image, or the like. In the specification, a form of the scanning image of the target object is not limited.

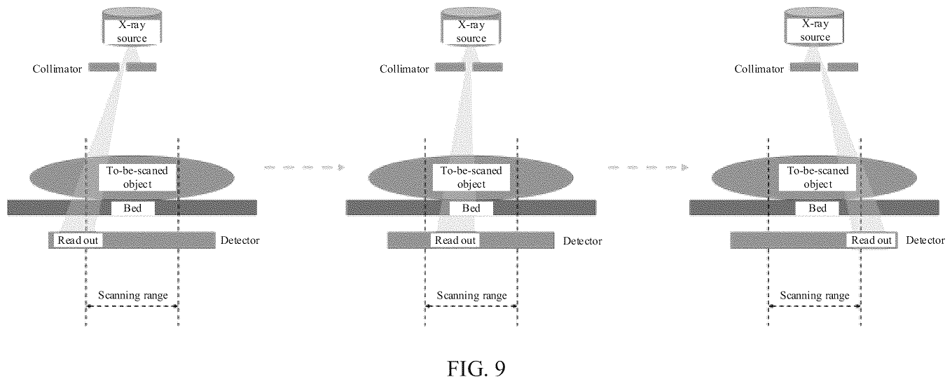

In an embodiment, referring to FIG. 9, an X-ray source may scan a to-be-scanned object through a hole or a gap in a collimating member when the collimating member moves. Since the hole or the gap of the collimating member shields remaining rays of the X-ray source, the detector can only read scanning data related to the hole or the gap of the collimating member. When the collimating member moves forward along a scanning direction, the X-ray source may complete scanning of the to-be-scanned object through the hole or the gap in the collimating member, and combine sub-images obtained by scanning to obtain a complete scanning image of the to-be-scanned object.

In an embodiment, either or both of the target object and a carrier that carries

the target object may be rotatable during scanning.

In an embodiment, a part of the detector is enabled to receive the imaging beam, and the part of the detector is irradiated by the imaging beam.

In an embodiment, the carrier that carries the target object may be not moved during scanning, and a receiving unit at an irradiated position of the carrier may be enabled or a signal at an irradiated position of the plate may be screened out, according to a position that receives the imaging beam or a relative motion between the collimating apparatus and the imaging source.

In a process of a scanning device scanning the target object, either or both of the target object and the carrier that carries the target object may be fixed in three-dimensional coordinates. The scanning device that scans the target object may serve as a movable member, and scan the target object.

In an embodiment, the scanning imaging source may be an imaging source of Fan-beam tomography or Cone-beam tomography.

In the foregoing generating method of the scanning image, different positions of the target object are scanned, respectively, by changing the passing region of the imaging beam emitted from the scanning imaging source, the plurality of scanning data of the target object are obtained, and the scanning image corresponding to the target object is obtained based on the plurality of scanning data of the target object.

During scanning, the object and the carrier that carries the target object may be still. Along a starting position of the scanning, the collimating apparatus with a narrow range may be added to the imaging source to limit the imaging beam to a narrow gap along an axial direction. At the same time, the detector only enables a unit which is irradiated to collect the imaging beam. In this way, a reading speed of the detector may be improved. In this state, a rack may rapidly rotate around once or multiple times to collect enough projection data for the image, and then the collimating apparatus may be moved to a position where a next narrow gap is adjacent to the start position of the scanning and maintain a same width of the gap for collection. An entire scanning range may be covered by continuous movement of the collimating apparatus and continuous collection of the plate. This may improve an imaging effect of scanning the target object by X-ray tomography, and a scanning length may meet a requirement.

In the foregoing embodiment, Fan-beam CT imaging with a high precision and a large range that is superior to Cone-beam CT imaging may be implemented in a case that the target object remains stationary (and alternatively, the imaging source and/or the detector also remain stationary). In addition, since the imaging source and/or the detector remain stationary, space of the device may be saved, and an electrical design, a motion track algorithm, and the like of the device may also be relatively simple.

In an exemplary embodiment, an imaging beam collimating apparatus may be disposed between the scanning imaging source and the target object, and the imaging beam collimating apparatus is configured to limit imaging beams of the scanning imaging source to a preset irradiation range along an axial direction. The imaging beam collimating apparatus may be in any type, for example, a collimator at an X-ray tube side of CT, an additional collimator, a movable stopper, a grating, or the like that is additionally mounted at a ray exit. In the method of the foregoing embodiment, scanning different positions of the target object, respectively, by changing the passing region of the imaging beam emitted from the scanning imaging source, and obtaining the plurality of scanning data of the target object may further include: irradiating the imaging beam emitted from the scanning imaging source to different positions of the target object by adjusting a position relationship between the imaging beam collimating apparatus and the scanning imaging source, scanning different positions of the target object, and obtaining the plurality of scanning data of the target object.

The position relationship may be a relative position relationship between the imaging beam collimating apparatus and the scanning imaging source, such as position 1 of the imaging beam collimating apparatus is corresponding to position 1 or position 2 of the scanning imaging source. Referring to FIG. 9, the position relationship between the imaging beam collimating apparatus and the scanning imaging source may change during a scanning process of the target object, i.e., the change of the position relationship may represent that a coordinate position of the imaging beam collimating apparatus changes relative to a coordinate position of the scanning imaging source.

An adjustable imaging beam collimating apparatus may be acquired, and the adjustable imaging beam collimating apparatus may change a direction and collimation of the imaging beam. A relative position relationship between the adjustable imaging beam collimating apparatus and the scanning imaging source may be adjusted, so that the imaging beam of the scanning imaging source may be irradiated to different positions of the target object by the imaging beam collimating apparatus. This may involve precise mechanical adjustment or an electronic control system. Then, it is ensured that the imaging beams are collimated, and scanning of different positions of the target object may be implemented by controlling a direction and an angle of emission of the scanning imaging source. By jointly adjusting the position relationship between the imaging beam collimating apparatus and the scanning imaging source, the plurality of scanning data of the target object may be obtained, so as to cover different positions of the target object.

In the present embodiment, the position relationship between the imaging beam collimating apparatus and the scanning imaging source may be adjusted, so that the scanning imaging beam may be irradiated at different positions of the target object. This adjustment with flexibility and precision may allow a system to obtain detailed scanning information of a plurality of different positions of the target object, thereby improving comprehensiveness and accuracy of scanning. By obtaining the plurality of scanning data of the target object, a structure and a feature of the target object may be fully understood, and more abundant data support may be provided for accurate analysis and application, which facilitates improving diagnostic accuracy and quality control effects in fields such as medical images and industrial detection. This method of position relationship adjustment may bring greater flexibility and information acquisition capability to an application of a scanning technology, and improve an overall effect.

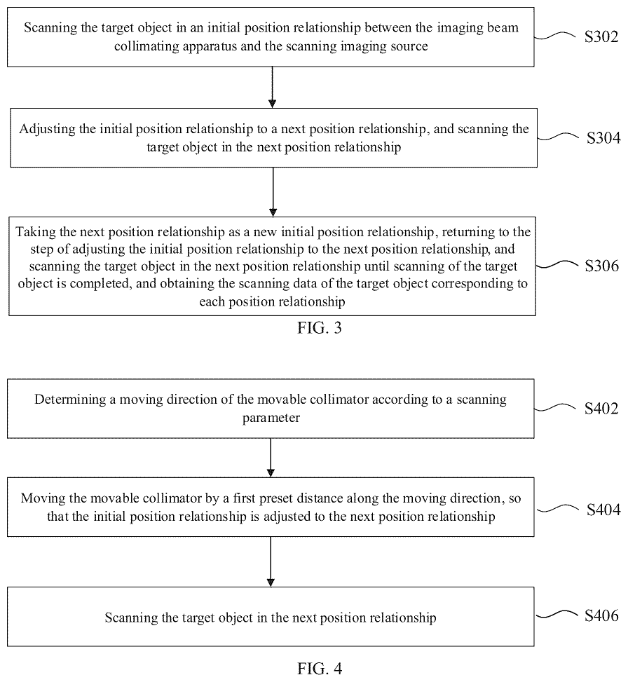

In an exemplary embodiment, referring to FIG. 3, irradiating the imaging beam emitted from the scanning imaging source to different positions of the target object by adjusting a position relationship between the imaging beam collimating apparatus and the scanning imaging source, scanning different positions of the target object, and obtaining the plurality of scanning data of the target object may further include step 302 to step 306.

Step 302 may include that scanning the target object in an initial position relationship between the imaging beam collimating apparatus and the scanning imaging source.

The initial position relationship may be a first position relationship between the imaging beam collimating apparatus and the scanning imaging source, i.e., a position relationship configured to perform a first scanning.

Specifically, it may require an accurate positioning system or a calibration process to ensure that the imaging beam collimating apparatus and the scanning imaging source are in a preset initial position relationship. The scanning imaging source may be started to emit the imaging beam, and the imaging beam collimating apparatus may ensure the direction and collimation of the imaging beam. After the imaging beam is irradiated to the target object, scanning data at the initial position may be obtained by detecting and recording information about interaction between the imaging beam and the target object, such as reflection and absorption. This process may involve application of the detector or a sensor to capture the interaction between the imaging beam and the target object. Finally, the obtained data may be processed and reconstructed to obtain a scanning result of the target object at the initial position, so as to form corresponding scanning data of the target object.

Step 304 may include that adjusting the initial position relationship to a next position relationship, and scanning the target object in the next position relationship.

The next position relationship may be a new relative position relationship between the imaging beam collimating apparatus and the scanning imaging source that is obtained by adjusting the relative position relationship between the imaging beam collimating apparatus and the scanning imaging source.

Specifically, the relative position of the imaging beam collimating apparatus and the scanning imaging source may be changed by an adjustable mechanism, which may include a mechanical moving system or an electronic control system to ensure accurate and repeatable position adjustment. Once the relative position relationship between the imaging beam collimating apparatus and the scanning imaging source is adjusted to the next position relationship, the scanning imaging source may be started, and it may be ensured that the imaging beam collimating apparatus is aligned with a new position of the target object. A scanning process similar to that at an initial position may be performed, and scanning data at the new position may be obtained by recording interaction information between the imaging beam and the target object. Finally, the obtained data may be processed and reconstructed to generate a scanning result of the target object in the next position relationship, and corresponding scanning data of the target object may be formed. This process may be cyclically executed, scanning data at a plurality of positions may be obtained, so as to implement comprehensive scanning of the target object.

Step 306 may include that taking the next position relationship as a new initial position relationship, returning to the step of adjusting the initial position relationship to the next position relationship, and scanning the target object in the next position relationship until scanning of the target object is completed, and obtaining the scanning data of the target object corresponding to each position relationship.

Specifically, after scanning corresponding to the next position relationship is completed, the obtained scanning data of the target object may be recorded and saved. Then, the next position relationship may be set as the new initial position relationship, and adjustment mechanism may be performed cyclically: the initial position relationship between the imaging beam collimating apparatus is adjusted to the next position relationship (i.e., from the next position relationship to a next position relationship). Then, the scanning may be performed, data may be collected and processed, and scanning data of the target object in the new position relationship may be obtained. This process may be repeated until scanning of the target object is completed, and finally scanning data corresponding to position relationships may be obtained. The scanning data corresponding to position relationships may be combined according to a preset rule, so that comprehensive scanning of the target object may be performed, and an image corresponding to comprehensive scanning may be obtained.

In the present embodiment, the target object may be scanned in the initial position relationship, and then different portions of the target object may be scanned step by step by adjusting the position relationship, to finally obtain scanning data of the target object corresponding to position relationships, so that comprehensive and detailed information of the target object may be obtained. This step-by-step scanning method may ensure that sufficient attention is paid to each position and improve the comprehensiveness and accuracy of the scanning. By taking the adjusted position relationship as a new initial relationship each time, a scanning procedure with seamless connection and continuity may be implemented, and consistency of scanning data may be ensured. This strategy may not only facilitate accurately restoring the structure of the target object, but also provide a more abundant basis for subsequent data processing, analysis, and diagnosis, thereby improving scanning effect and application feasibility in fields such as medical images and industrial detection.

In an embodiment, the imaging beam collimating apparatus may include a movable collimator, and adjusting the initial position relationship to the next position relationship, and scanning the target object in the next position relationship may further include: moving the movable collimator, so that the initial position relationship is adjusted to the next position relationship; and scanning the target object in the next position relationship.

In an exemplary embodiment, referring to FIG. 4, the imaging beam collimating apparatus may include a movable collimator. Adjusting the initial position relationship to the next position relationship, and scanning the target object in the next position relationship may further include step 402 to step 406.

Step 402 may include that determining a moving direction of the movable collimator according to a scanning parameter.

The scanning parameter may include a scanning direction of the target object. The scanning direction may be a direction along which the scanning device scans the target object. The direction may be defined from the start of scanning.

Specifically, a geometric structure and a scanning requirement of the target object may be analyzed to obtain an association algorithm or an association rule, and the scanning direction of the target object may be associated with the moving direction of the movable collimator. This may involve a mathematical model, geometric calculation, or a machine learning method. When a direction relationship is established, a system may correspondingly adjust the moving direction of the movable collimator by monitoring the scanning direction of the target object in real time, so as to ensure that the imaging beam can effectively cover different regions of the target object.

Step 404 may include that moving the movable collimator by a first preset distance along the moving direction, so that the initial position relationship is adjusted to the next position relationship.

The movable collimator may be a collimator that the imaging beam collimating apparatus is movable.

The first preset distance may be a distance for each movement of the movable collimator.

Specifically, the movable collimator may be moved by the first preset distance towards a required moving direction via controlling a mechanical system or an electronic system of the movable collimator. This may require real-time monitoring and feedback mechanisms to ensure accurate moving distance. After the movement is completed, the position relationship between the imaging beam collimating apparatus and the scanning imaging source may be adjusted from the initial position relationship to the next position relationship.

Step 406 may include that scanning the target object in the next position relationship.

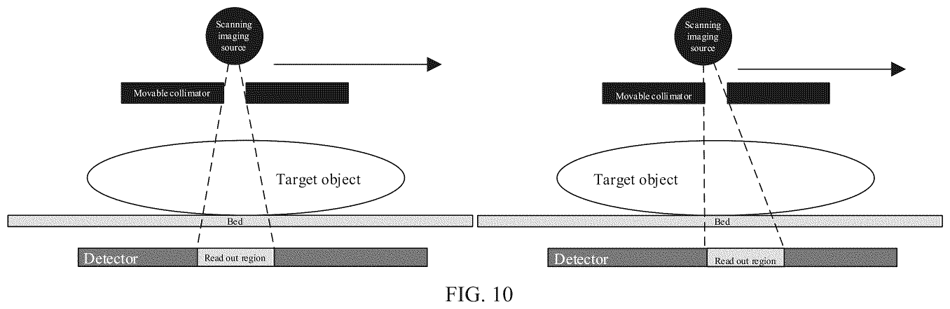

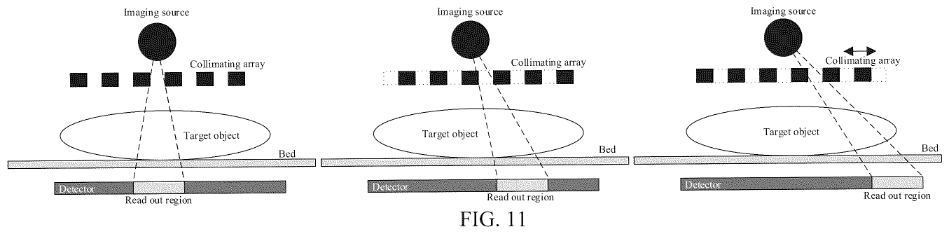

Specifically, it may be ensured that the movable collimator and the scanning imaging source have been adjusted to the new position relationship, and the scanning imaging source may be started to emit the imaging beam. The movable collimator may ensure the direction and collimation of the imaging beam. After the imaging beam is irradiated to the target object, the scanning data in the new position relationship may be obtained by detecting and recording information about interaction between the imaging beam and the target object, such as reflection and absorption. This may involve application of the detector or the sensor. The obtained data may be processed and reconstructed to generate a scanning result of the target object in the next position relationship, so as to form corresponding scanning data of the target object. After the scanning ends, the current next position relationship may be set to a new initial position relationship, so as to prepare for a next scanning. This process may be cyclically performed until scanning of the target object is completed, to obtain scanning data corresponding to position relationships. Referring to FIG. 10, in a case that the movable collimator moves along the scanning direction, the scanning imaging source may complete comprehensive scanning of the scanned object through the hole or the gap of the movable collimator.

In the present embodiment, the moving direction of the movable collimator may be determined according to the scanning direction of the target object, and the system may implement more intelligent and targeted scanning. The movable collimator may be moved by the first preset distance along the moving direction, a scanning focal point may be effectively adjusted, so as to ensure that the imaging beam can be accurately irradiated to different positions of the target object. Such accurate adjustment can improve positioning accuracy of the scanning and scanning quality. In the next position relationship, the target object may be scanned to fully cover the adjusted region, so as to obtain more comprehensive and detailed scanning data. This step may facilitate improving scanning efficiency, reducing unnecessary repeated scanning, and ensuring that a high-quality scanning image of the target object is obtained.

In an embodiment, the imaging beam collimating apparatus may include a movable collimating array, and adjusting the initial position relationship to the next position relationship, and scanning the target object in the next position relationship may further includes: moving the movable collimating array, so that the initial position relationship is adjusted to the next position relationship; and scanning the target object in the next position relationship.

In an exemplary embodiment, referring to FIG. 5, the imaging beam collimating apparatus may include a collimating array. Adjusting the initial position relationship to the next position relationship, and scanning the target object in the next position relationship may further include step 502 to step 506.

Step 502 may include that determining a first moving direction or a second moving direction of the movable collimating array according to a scanning parameter. The first moving direction is opposite to the second moving direction.

The movable collimating array may be a movable collimating gating. The scanning parameter may include scanning times of the target object.

Specifically, the first moving direction or the second moving direction of the movable collimating array may be determined according to the scanning times of the target object, and a relationship between the scanning times and the moving direction may need to be defined. By presetting a rule or an algorithm, it is determined, according to the scanning requirement and the geometric structure of the target object, when the array should be moved along the first moving direction or the second moving direction, and factors need to be considered such as an optimal scanning path, coverage, and the like. Then, according to actual scanning times and the preset rule, the system can automatically select an appropriate moving direction to ensure that all regions of the target object are covered during the entire scanning process. This process may be implemented by programming to implement an automated system that dynamically adjusts the moving direction according to the scanning times.

Step 504 may include that moving the movable collimating array by a second preset distance along the first moving direction or the second moving direction, so that the initial position relationship is adjusted to the next position relationship.

The second preset distance may be a distance for each movement of the movable collimating array.

Specifically, when it is determined whether the current selected moving direction is the first moving direction or the second moving direction, the movable collimating array may be moved by the second preset distance along the selected moving direction via controlling a mechanical system or an electronic system of the movable collimating array. This may require real-time monitoring and feedback mechanisms to ensure accurate moving distance. After the movement is completed, the position relationship between the imaging beam collimating apparatus and the scanning imaging source may be adjusted from the initial position relationship to the next position relationship.

Step 506 may include that scanning the target object in the next position relationship.

Specifically, it may be ensured that the movable collimating array and the scanning imaging source have been adjusted to the new position relationship, and the scanning imaging source may be started to emit the imaging beam. The movable collimating array may ensure the direction and collimation of the imaging beam. After the imaging beam is irradiated to the target object, the scanning data in the new position relationship may be obtained by detecting and recording information about interaction between the imaging beam and the target object, such as reflection and absorption. This may involve application of the detector or the sensor. The obtained data may be processed and reconstructed to generate a scanning result of the target object in the next position relationship, so as to form corresponding scanning data of the target object. After the scanning ends, the current next position relationship may be set to a new initial position relationship, so as to prepare for a next scanning. This process may be cyclically performed until scanning of the target object is completed, to obtain scanning data corresponding to position relationships. Referring to FIG. 11, in a case that the movable collimating array moves along the first moving direction or the second moving direction, the scanning imaging source may complete comprehensive scanning of the scanned object through the hole or the gap of the movable collimating array.

In the present embodiment, the first moving direction or the second moving direction of the movable collimating array may be determined according to the scanning times of the target object, and the system may implement targeted scanning. The first moving direction is opposite to the second moving direction, so that scanning along different directions may be complementary and cover more surfaces of the target object. The movable collimating array may be moved by the second preset distance along in the first moving direction or the second moving direction, so as to adjust the position relationship and scan the target object in the next position relationship. This multi-direction movement and adjustment strategy may effectively improve coverage and comprehensiveness of the scanning, and facilitate capturing detailed information along various directions of the target object. Such a scanning method may optimize image reconstruction and analysis, and improve a stereo sense and accuracy of data.

In an embodiment, the scanning imaging source may include an imaging source with a fly focus, and adjusting the initial position relationship to the next position relationship, and scanning the target object in the next position relationship may further include: adjusting a focus of the imaging source with the fly focus, so that the initial position relationship is adjusted to the next position relationship; and scanning the target object in the next position relationship.

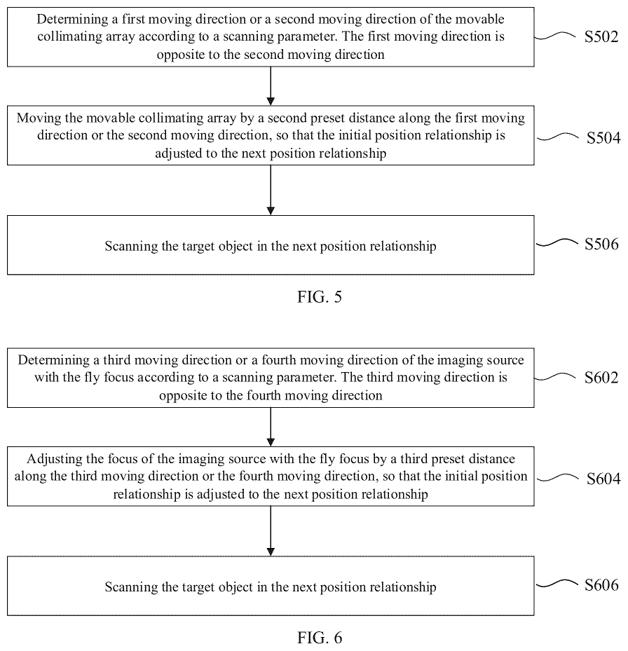

In an exemplary embodiment, referring to FIG. 6, the scanning imaging source may include an imaging source with a fly focus. Adjusting the initial position relationship to the next position relationship, and scanning the target object in the next position relationship may further include step 602 to step 606.

Step 602 may include that determining a third moving direction or a fourth moving direction of the imaging source with the fly focus according to a scanning parameter. The third moving direction is opposite to the fourth moving direction.

The imaging source with the fly focus may be an imaging source whose focus is rapidly converted at two different positions on a target surface. The scanning parameter may include scanning times of the target object.

Specifically, the third moving direction or the fourth moving direction of the imaging source with the fly focus may be determined according to the scanning times of the target object, and a relationship between the scanning times and the moving direction may need to be defined. By presetting a rule or an algorithm, it is determined, according to the scanning requirement and the geometric structure of the target object, when the imaging source with the fly focus should be moved along the third moving direction or the fourth moving direction, and factors need to be considered such as an optimal scanning path, coverage, and the like. Then, according to actual scanning times and the preset rule, the system can automatically select an appropriate moving direction to ensure that all regions of the target object are covered during the entire scanning process. This process may be implemented by programming to implement an automated system that dynamically adjusts the moving direction according to the scanning times.

Step 604 may include that adjusting the focus of the imaging source with the fly focus by a third preset distance along the third moving direction or the fourth moving direction, so that the initial position relationship is adjusted to the next position relationship.

The third preset distance may be a distance for each movement of the imaging source with the fly focus.

Specifically, when it is determined whether the current selected moving direction is the third moving direction or the fourth moving direction, the imaging source with the fly focus may be moved by the third preset distance along the selected moving direction via controlling a mechanical system or an electronic system of the imaging source with the fly focus. This may require real-time monitoring and feedback mechanisms to ensure accurate moving distance. After the movement is completed, the position relationship between the imaging beam collimating apparatus and the scanning imaging source may be adjusted from the initial position relationship to the next position relationship.

Step 606 may include that scanning the target object in the next position relationship.

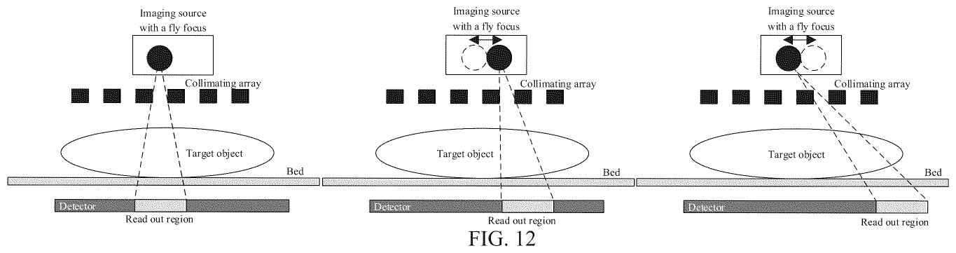

Specifically, it may be ensured that the imaging source with the fly focus and the imaging beam collimating apparatus have been adjusted to the new position relationship, and the imaging source with the fly focus may be started to emit the imaging beam. The imaging beam collimating apparatus may ensure the direction and collimation of the imaging beam. After the imaging beam is irradiated to the target object, the scanning data in the new position relationship may be obtained by detecting and recording information about interaction between the imaging beam and the target object, such as reflection and absorption. This may involve application of the detector or the sensor. The obtained data may be processed and reconstructed to generate a scanning result of the target object in the next position relationship, so as to form corresponding scanning data of the target object. After the scanning ends, the current next position relationship may be set to a new initial position relationship, so as to prepare for a next scanning. This process may be cyclically performed until scanning of the target object is completed, to obtain scanning data corresponding to position relationships. Referring to FIG. 12, in a case that the imaging source with the fly focus moves along the third moving direction or the fourth moving direction, the imaging source with the fly focus may complete comprehensive scanning of the scanned object through the hole or the gap of the movable collimating apparatus.

In the present embodiment, the third moving direction or the fourth moving direction of the imaging source with the fly focus may be determined according to the scanning times of the target object, and the system may implement more intelligent and customized movement strategy of the imaging source. The third moving direction is opposite to the fourth moving direction, so that scanning along different directions may be complementary and improve comprehensiveness of irradiation of the imaging source. The imaging source with the fly focus may be moved by the third preset distance along in the third moving direction or the fourth moving direction, so as to adjust the position relationship and scan the target object in the next position relationship. Such movement strategy of the imaging source may optimize an irradiation angle to obtain more stereoscopic and detailed scanning data, which facilitates improving image quality and resolution.

In an embodiment, the scanning imaging source comprises an imaging source array, and adjusting the initial position relationship to the next position relationship, and scanning the target object in the next position relationship may further include: switching a current scintillating imaging source to a next scintillating imaging source according to a switching direction, so that the initial position relationship is adjusted to the next position relationship; and scanning the target object in the next position relationship.

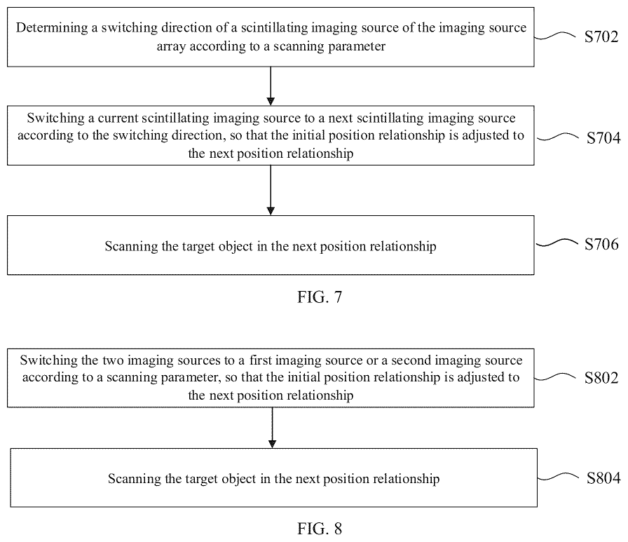

In an exemplary embodiment, referring to FIG. 7, the scanning imaging source may include an imaging source array. Adjusting the initial position relationship to the next position relationship, and scanning the target object in the next position relationship may further include step 702 to step 706.

Step 702 may include that determining a switching direction of a scintillating imaging source of the imaging source array according to a scanning parameter.

The imaging source array may be an array of a plurality of imaging sources. The scanning parameter may include a scanning direction of the target object.

Specifically, the switching direction of a scintillating imaging source of the imaging source array may be determined according to the scanning direction of the target object, and a relationship between the scanning direction and the switching direction of the imaging source may need to be learned. The geometric structure and the scanning requirement of the target object may be analyzed, and a switching algorithm of the scintillating imaging source or a switching rule of the scintillating imaging source may be applied to associate the scanning direction of the target object with the switching direction of the imaging source. This may involve a mathematical model, geometric calculation, or a machine learning method. When a direction relationship is established, a system may correspondingly adjust the switching direction of the scintillating imaging source in the imaging source array by monitoring the scanning direction of the target object in real time, so as to ensure that the change of the imaging source can effectively cooperate with the scanning. This process may require real-time feedback and dynamic adjustment to ensure the timeliness and effectiveness of switching of the imaging source along different scanning directions.

Step 704 may include that switching a current scintillating imaging source to a next scintillating imaging source according to the switching direction, so that the initial position relationship is adjusted to the next position relationship.

Specifically, when a mechanism that clearly defines the switching direction is determined, it is ensured that the current scintillating imaging source is switched to the next scintillating imaging source along the selected switching direction by controlling the electronic or mechanical system in the imaging source array. This may require real-time monitoring and feedback mechanisms to ensure accurate switching of the imaging source. After the switching is completed, the position relationship between the imaging beam collimating apparatus and the scanning imaging source may be adjusted from the initial position relationship to the next position relationship.

Step 706 may include that scanning the target object in the next position relationship.

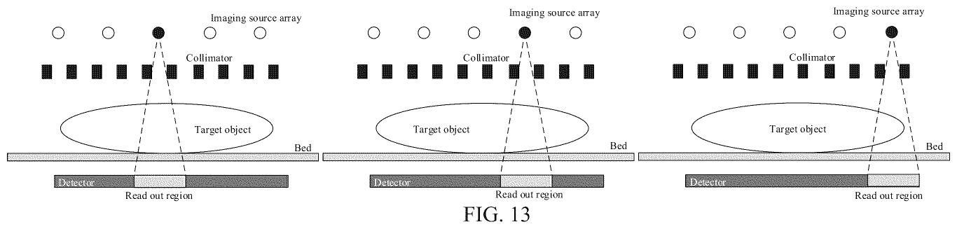

Specifically, it may be ensured that the imaging source array and the imaging beam collimating apparatus have been adjusted to the new position relationship, and the imaging source array may be started to emit the imaging beam. The imaging beam collimating apparatus may ensure the direction and collimation of the imaging beam. After the imaging beam is irradiated to the target object, the scanning data in the new position relationship may be obtained by detecting and recording information about interaction between the imaging beam and the target object, such as reflection and absorption. This may involve application of the detector or the sensor. The obtained data may be processed and reconstructed to generate a scanning result of the target object in the next position relationship, so as to form corresponding scanning data of the target object. After the scanning ends, the current next position relationship may be set to a new initial position relationship, so as to prepare for a next scanning. This process may be cyclically performed until scanning of the target object is completed, to obtain scanning data corresponding to position relationships. Referring to FIG. 13, in a case that the current scintillating imaging source is switched to a next scintillating imaging source during switching direction of the imaging source array, the scanning imaging source may complete comprehensive scanning of the scanned object through the hole or the gap of the movable collimating apparatus.

In the present embodiment, the switching direction of the scintillating imaging source of the imaging source array may be determined according to the scanning direction of the target object, and the system may implement more intelligent and targeted light switching strategy of the imaging source. The current scintillation imaging source may be switched to a next scintillation imaging source along the switching direction, so that a change of the imaging source during a scanning process can effectively cooperate with scanning. Such a strategy may facilitate improving imaging beam lighting quality in different regions of the target object, and optimizing image contrast and clarity. The target object may be scanned in the next location relationship to ensure sufficient radiographic illumination and clearer scanning images. This intelligent imaging source switching manner can meet requirements of different scanning scenarios, and improve scanning effects and data quality in fields such as medical images and industrial detection.

In an exemplary embodiment, referring to FIG. 8, the scanning imaging source may include two imaging sources. Adjusting the initial position relationship to the next position relationship, and scanning the target object in the next position relationship may further include step 802 to step 804.

Step 802 may include that switching the two imaging sources to a first imaging source or a second imaging source according to a scanning parameter, so that the initial position relationship is adjusted to the next position relationship.

The two imaging sources may be a scanning imaging source with two imaging sources. The scanning parameter may include scanning times of the target object.

Specifically, in a case that a relationship between scanning times and switching of the imaging source is defined, the geometric structure and the scanning requirement of the target object may be considered, and it is determined when the first imaging source or the second imaging source should be switched to, via a preset switching rule of two imaging sources or a preset switching algorithm of two imaging sources. According to actual scanning times and the preset rule, the system can automatically select an appropriate imaging source to ensure effective cooperation throughout the scanning process. Switching between the two imaging sources may be implemented by controlling the electronic or mechanical systems of the two imaging sources. After the switching is complete, the position relationship between the imaging beam collimating apparatus and the scanning imaging source may be adjusted from the initial position relationship to the next position relationship.

Step 804 may include that scanning the target object in the next position relationship.

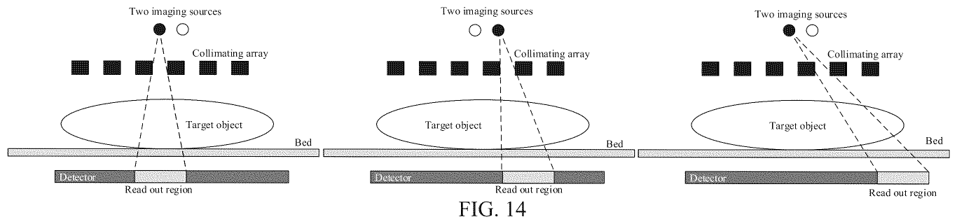

Specifically, it may be ensured that the two imaging sources and the imaging beam collimating apparatus have been adjusted to the new position relationship, and the two imaging sources may be started to emit the imaging beam. The imaging beam collimating apparatus may ensure the direction and collimation of the imaging beam. After the imaging beam is irradiated to the target object, the scanning data in the new position relationship may be obtained by detecting and recording information about interaction between the imaging beam and the target object, such as reflection and absorption. This may involve application of the detector or the sensor. The obtained data may be processed and reconstructed to generate a scanning result of the target object in the next position relationship, so as to form corresponding scanning data of the target object. After the scanning ends, the current next position relationship may be set to a new initial position relationship, so as to prepare for a next scanning. This process may be cyclically performed until scanning of the target object is completed, to obtain scanning data corresponding to position relationships. Referring to FIG. 14, in a case that the two imaging sources are switched to the first imaging source or the second imaging source according to scanning times of the target object, the scanning imaging source may complete comprehensive scanning of the scanned object through the hole or the gap of the movable collimating apparatus.

In the present embodiment, two imaging sources may be switched to the first imaging source or the second imaging source according to scanning times of the target object, so that the system can select a proper imaging source according to a specific scanning requirement, thereby implementing a more flexible and intelligent imaging beam lighting strategy. The first imaging source and the second imaging source may be mutually exclusive imaging sources, so that only one imaging source is in operation at a same moment, and interference between imaging sources may be prevented. The target object may be scanned in the next position relationship, more comprehensive and multi-angle information about the target object may be obtained by features of different imaging sources. This manner of dynamically switching imaging sources may improve a system capability of adapting to different scanning scenarios.

It should be understood that, although steps in the flowchart related to the foregoing embodiments are sequentially displayed according to an instruction of an arrow, these steps are not necessarily sequentially performed according to the instruction of the arrow. Unless expressly stated in the specification, these steps are not performed in a strict order, and these steps may be performed in another order. In addition, at least a part of steps in the flowchart involved in the foregoing embodiments may include multiple steps or multiple phases. These steps or phases are not necessarily performed at a same moment, but may be performed at different moments. An execution sequence of these steps or phases is not necessarily performed sequentially, but may be performed alternately with other steps or alternately with at least a part of steps or phases in other steps.

Based on a same invention concept, an embodiment of the present disclosure may further provide a generating apparatus of a scanning image configured to implement the foregoing generating method of the scanning image. An implementation solution provided by the generating apparatus may be similar to an implementation solution described in the foregoing method. Therefore, a specific limitation in one or more embodiments of the generating apparatus of the scanning image provided below may refer to the foregoing limitation in the generating method of the scanning image. Details are not described herein again.

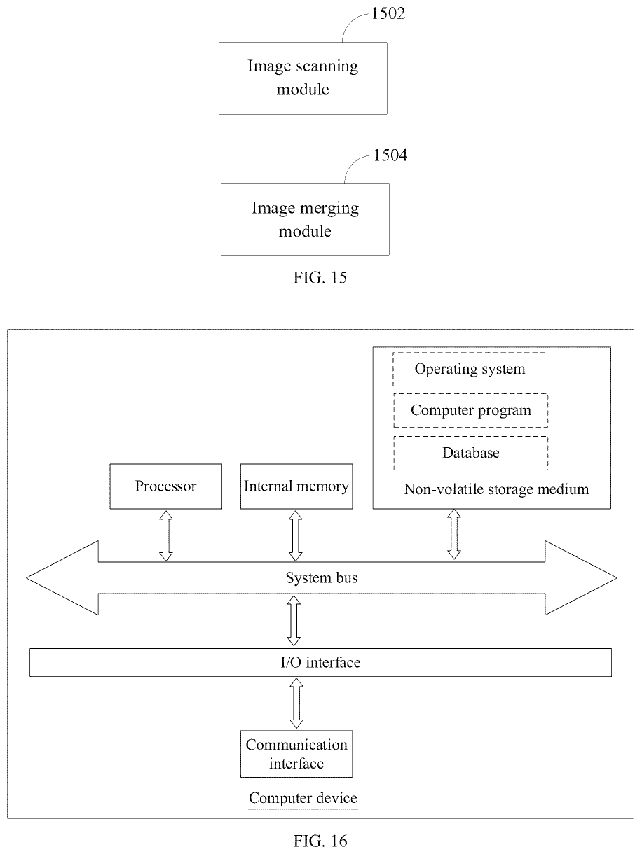

In an exemplary embodiment, referring to FIG. 15, a generating apparatus of a scanning image is provided, including an image scanning module 1502 and an image merging module 1504.

The image scanning module 1502 is configured for scanning different positions of a target object, respectively, by changing a passing region of an imaging beam emitted from a scanning imaging source, obtaining a plurality of scanning data of the target object.

The image merging module 1504 is configured for obtaining a scanning image corresponding to the target object based on the plurality of scanning data of the target object.

In an embodiment, the image scanning module 1502 is further configured for irradiating the imaging beam emitted from the scanning imaging source to different positions of the target object by adjusting a position relationship between the imaging beam collimating apparatus and the scanning imaging source, scanning different positions of the target object, and obtaining the plurality of scanning data of the target object.

In an embodiment, the image scanning module 1502 is further configured for scanning the target object in an initial position relationship between the imaging beam collimating apparatus and the scanning imaging source, adjusting the initial position relationship to a next position relationship, and scanning the target object in the next position relationship, and taking the next position relationship as a new initial position relationship, returning to the step of adjusting the initial position relationship to the next position relationship, and scanning the target object in the next position relationship until scanning of the target object is completed, and obtaining the scanning data of the target object corresponding to each position relationship.

In an embodiment, the image scanning module 1502 is further configured for determining a moving direction of a movable collimator according to a scanning direction of the target object, moving the movable collimator by a first preset distance along the moving direction, so that the initial position relationship is adjusted to the next position relationship, and scanning the target object in the next position relationship.

In an embodiment, the image scanning module 1502 is further configured for determining a first moving direction or a second moving direction of a movable collimating array according to scanning times of the target object, moving the movable collimating array by a second preset distance along the first moving direction or the second moving direction, so that the initial position relationship is adjusted to the next position relationship, and scanning the target object in the next position relationship. The first moving direction is opposite to the second moving direction.

In an embodiment, the image scanning module 1502 is further configured for determining a third moving direction or a fourth moving direction of an imaging source with a fly focus according to scanning times of the target object, adjusting the focus of the imaging source with the fly focus by a third preset distance along the third moving direction or the fourth moving direction, so that the initial position relationship is adjusted to the next position relationship, and scanning the target object in the next position relationship. The third moving direction is opposite to the fourth moving direction.

In an embodiment, the image scanning module 1502 is further configured for determining a switching direction of a scintillating imaging source of the imaging source array according to a scanning direction of the target object, switching a current scintillating imaging source to a next scintillating imaging source according to the switching direction, so that the initial position relationship is adjusted to the next position relationship, and scanning the target object in the next position relationship.

In an embodiment, the image scanning module 1502 is further configured for switching the two imaging sources to a first imaging source or a second imaging source according to scanning times of the target object, so that the initial position relationship is adjusted to the next position relationship, and scanning the target object in the next position relationship.

All modules in the foregoing generating apparatus of the scanning image may be implemented in whole or in part by software, hardware, and a combination thereof. The foregoing modules may be embedded in or independent of a processor in a computer device in a hardware form, or may be stored in a memory in the computer device in a software form, so that the processor may invoke to execute an operation corresponding to the foregoing modules.

In an exemplary embodiment, a generating system of a scanning image is provided, including a scanning device and a computer device. The scanning device is configured for scanning different positions of a target object, respectively, by changing a passing region of an imaging beam emitted from a scanning imaging source, and obtaining a plurality of scanning data of the target object. The computer device is configured for obtaining a scanning image corresponding to the target object based on the plurality of scanning data of the target object.

In an exemplary embodiment, a generating device of a scanning image is provided, including a scanning imaging source, an imaging beam collimating apparatus, a detector, and a processor. The imaging beam collimating apparatus is configured for changing a passing region of an imaging beam emitted from the scanning imaging source, so that the imaging beam scans different positions of a target object, respectively. The detector is configured for receiving the imaging beam, and obtaining a plurality of scanning data corresponding to the different positions of the target object based on the imaging beam. The processor is configured for obtaining a scanning image corresponding to the target object based on the plurality of scanning data of the target object.

In an exemplary embodiment, a radiation delivery system is provided, including the generating device of the scanning image in the foregoing embodiment and a radiation delivery device. The radiation delivery device is configured for performing radiation delivery to the target object based on the scanning image of the target object.

As an example, the radiation delivery may include radiation therapy, radiation processing, or the like.

As an example, performing radiation delivery to the target object based on the scanning image of the target object may include: planning (such as a desired radiation dose, a desired radiation location, or the like determined by artificiality or a computing device based on the scanning image of the target object) and executing radiation delivery based on the scanning image of the target object acquired before or during radiation delivery.

Since beneficial effects of the generating device of the scanning image in the present disclosure, a position of the target object may keep unchanged during the generating of the scanning image and radiation delivery. Therefore, there is no deviation between an actual position of radiation delivery and a position based on image planning due to movement, and no additional motion control device or motion control algorithm is required to ensure that a position of the radiation delivery is consistent with an imaging position. In addition, since the target object is no need to move, it may also be ensured that the target object does not collide with the radiation delivery device and the generating device of the scanning image.

In an exemplary embodiment, a computer device is provided. The computer device may be a server, and an internal structure diagram of the computer device may refer to FIG. 16. The computer device includes a processor, a memory, an Input/Output (I/O) interface, and a communication interface. The processor, the memory, and the input/output interface are connected by a system bus, and the communications interface is connected to the system bus by the input/output interface. The processor of the computer device is configured to provide a computing and control capability. The memory of the computer device includes a non-volatile storage medium and an internal memory. The non-volatile storage medium stores an operating system, a computer program, and a database. The internal memory provides an environment for running the operating system and the computer program in the non-volatile storage medium. The database of the computer device is configured to store server data. The Input/Output interface of the computer device is configured to exchange information between the processor and an external device. The communication interface of the computer device is configured to communicate with an external terminal by a network connection. The computer program is executed by the processor to implement the generating method of the scanning image.

One skilled in the art may understand that the structure shown in FIG. 16 is merely a block diagram of some structures related to the solutions of the present disclosure, and does not constitute a limitation on a computer device to which the solutions of the present disclosure are applied. A specific computer device may include more or fewer components than those shown in the figure, combine some components, or have different component arrangements.

In an embodiment, a computer device is further provided, including a memory and a processor. The memory stores a computer program, and the processor implements steps in the foregoing method embodiments when executing the computer program.

In an embodiment, a computer-readable storage medium is provided, and a computer program is stored in the computer-readable storage medium. When being executed by a processor, the computer program implements the steps in the foregoing method embodiments.

In an embodiment, a computer program product or a computer program is provided. The computer program product or the computer program includes a computer instruction, and the computer instruction is stored in a computer-readable storage medium. A processor of a computer device reads the computer instruction from the computer-readable storage medium, and the processor executes the computer instruction, so that the computer device performs the steps in the foregoing method embodiments.

It should be noted that user information (including but not limited to user equipment information, user personal information, or the like) and data (including but not limited to data used for analysis, stored data, and displayed data) involved in the present disclosure are information and data those are authorized by the user or those are fully authorized by each party, and collection, use, and processing of related data need to comply with related regulations.

One skilled in the art may understand that all or a part of the processes in the methods in the foregoing embodiments may be implemented by a computer program instructing related hardware. The computer program may be stored in a non-volatile computer-readable storage medium. When the computer program is executed, the processes in the foregoing methods embodiments may be included. Any reference to a memory, a database, or another medium used in the embodiments provided in the present disclosure may include at least one of a non-volatile memory or a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto resistive Random Access Memory (MRAM), a Ferroelectric Random Access Memory (FRAM), a Phase Change Memory (PCM), a graphene memory, or the like. The volatile memory may include a Random Access Memory (RAM), an external cache, or the like. As an illustration and not a limitation, the RAM may be in multiple forms, such as a Static Random Access Memory (SRAM) or a Dynamic Random Access Memory (DRAM). The databases involved in the embodiments provided in the present disclosure may include at least one of a relational database or a non-relational database. The non-relational database may include a distributed database based on a block chain, or the like, which is not limited thereto. The processor in the embodiments provided in present disclosure may be a general-purpose processor, a central processing unit, a graphics processor, a digital signal processor, a programmable logic device, a data processing logic device based on quantum computing, or the like, which is not limited thereto.

All the technical features in the foregoing embodiments may be any combination. To make the description brief, all possible combinations of the technical features in the foregoing embodiments are not described. However, as long as there is no contradiction between the combinations of the technical features, it should be considered as the scope described in this specification.

The foregoing embodiments represent only several implementation manners of this application, and descriptions thereof are relatively specific and detailed, but may not be construed as a limitation on the scope of this application. It should be noted that a person of ordinary skill in the art may make some modifications and improvements without departing from the concept of this application, which are within the protection scope of this application. Therefore, the protection scope of this application shall be subject to the appended claims.

What is claimed is:

1. A generating method of a scanning image, comprising:
scanning different positions of a target object, respectively, by changing a passing region of an imaging beam emitted from a scanning imaging source, and obtaining a plurality of scanning data of the target object; and
obtaining a scanning image corresponding to the target object based on the plurality of scanning data of the target object.

2. The method of claim 1, wherein a detector configured for receiving the imaging beam is rotatable during scanning.

3. The method of claim 2, wherein a part of the detector is enabled to receive the imaging beam, and the part of the detector is irradiated by the imaging beam.

4. The method of claim 1, wherein either or both of the target object and a carrier that carries the target object remain at a fixed position during scanning.

5. The method of claim 1, wherein the scanning imaging source is an imaging beam source of Fan-beam tomography or Cone-beam tomography.

6. The method of claim 1, wherein an imaging beam collimating apparatus is disposed between the scanning imaging source and the target object, the imaging beam collimating apparatus is configured to limit the imaging beam of the scanning imaging source to a preset irradiation range along an axial direction.

7. The method of claim 6, wherein scanning different positions of the target object, respectively, by changing the passing region of the imaging beam emitted from the scanning imaging source, and obtaining the plurality of scanning data of the target object further comprises:
irradiating the imaging beam emitted from the scanning imaging source to different positions of the target object by adjusting a position relationship between the imaging beam collimating apparatus and the scanning imaging source, scanning different positions of the target object, and obtaining the plurality of scanning data of the target object.

8. The method of claim 7, wherein irradiating the imaging beam emitted from the scanning imaging source to different positions of the target object by adjusting a position relationship between the imaging beam collimating apparatus and the scanning imaging source, scanning different positions of the target object, and obtaining the plurality of scanning data of the target object further comprises:
scanning the target object in an initial position relationship between the imaging beam collimating apparatus and the scanning imaging source;
adjusting the initial position relationship to a next position relationship, and scanning the target object in the next position relationship; and
taking the next position relationship as a new initial position relationship, returning to the step of adjusting the initial position relationship to the next position relationship, and scanning the target object in the next position relationship until scanning of the target object is completed, and obtaining the scanning data of the target object corresponding to each position relationship.

9. The method of claim 8, wherein the imaging beam collimating apparatus comprises a movable collimator, and adjusting the initial position relationship to the next position relationship, and scanning the target object in the next position relationship further comprises:
moving the movable collimator, so that the initial position relationship is adjusted to the next position relationship; and
scanning the target object in the next position relationship.

10. The method of claim 8, wherein the imaging beam collimating apparatus comprises a movable collimating array, and adjusting the initial position relationship to the next position relationship, and scanning the target object in the next position relationship further comprises:
moving the movable collimating array, so that the initial position relationship is adjusted to the next position relationship; and
scanning the target object in the next position relationship.

11. The method of claim 8, wherein the scanning imaging source comprises an imaging source with a fly focus, and adjusting the initial position relationship to the next position relationship, and scanning the target object in the next position relationship further comprises:
adjusting a focus of the imaging source with the fly focus, so that the initial position relationship is adjusted to the next position relationship; and
scanning the target object in the next position relationship.

12. The method of claim 8, wherein the scanning imaging source comprises an imaging source array, and adjusting the initial position relationship to the next position relationship, and scanning the target object in the next position relationship further comprises:
switching a current scintillating imaging source to a next scintillating imaging source according to a switching direction, so that the initial position relationship is adjusted to the next position relationship; and
scanning the target object in the next position relationship.

13. The method of claim 8, wherein the scanning imaging source comprises two imaging sources, and adjusting the initial position relationship to the next position relationship, and scanning the target object in the next position relationship further comprises:
switching the two imaging sources to a first imaging source or a second imaging source according to a scanning parameter, so that the initial position relationship is adjusted to the next position relationship; and
scanning the target object in the next position relationship.

14. A generating system of a scanning image, comprising a scanning device and a computer device,
wherein the scanning device is configured for scanning different positions of a target object, respectively, by changing a passing region of an imaging beam emitted from a scanning imaging source, and obtaining a plurality of scanning data of the target object; and
the computer device is configured for obtaining a scanning image corresponding to the target object based on the plurality of scanning data of the target object.

15. A generating device of a scanning image, comprising a scanning imaging source, an imaging beam collimating apparatus, a detector, and a processor,
wherein the imaging beam collimating apparatus is configured for changing a passing region of an imaging beam emitted from the scanning imaging source, so that the imaging beam scans different positions of a target object, respectively;
the detector is configured for receiving the imaging beam, and obtaining a plurality of scanning data corresponding to the different positions of the target object based on the imaging beam; and
the processor is configured for obtaining a scanning image corresponding to the target object based on the plurality of scanning data of the target object.

16. A radiation delivery system, comprising the generating device of the scanning image according to claim 14 and a radiation delivery device,
wherein the radiation delivery device is configured for performing radiation delivery to the target object based on the scanning image of the target object.

17. A computer-readable storage medium, on which a computer program is stored, wherein the computer program is executed by a processor to implement steps of the method of claim 1.

18. The computer-readable storage medium of claim 17, wherein a detector configured for receiving the imaging beam is rotatable during scanning.

19. The computer-readable storage medium of claim 17, wherein the target object remains at a fixed position during scanning.

20. The computer-readable storage medium of claim 17, wherein the scanning imaging source is an imaging beam source of Fan-beam tomography or Cone-beam tomography.
